SEQUENCE LISTING (14 sequences)

SEQ ID NO: 1 - DNA, 57 bp, Artificial
  Primer
taattttgtttaactttaagaaggagatataccatggtggacgccttcgtgggtacc

SEQ ID NO: 2 - DNA, 49 bp, Artificial
  Primer
tttaactttaagaaggagatataccatggtggacgccttcgtgggtacc

SEQ ID NO: 3 - DNA, 42 bp, Artificial
  Primer
cgtttagaggccccaaggggggtcatgcctgtttctcgtaag

SEQ ID NO: 4 - DNA, 51 bp, Artificial
  Primer
cgaactgcgggtggctccaagcgcttgcctgtttctcgtaagtacgagtgc

SEQ ID NO: 5 - DNA, 63 bp, Artificial
  Primer
cgtttagaggccccaagggggggagtagaatgttaaggattagtcatgcctgtttctcgtaag

SEQ ID NO: 6 - DNA, 75 bp, Artificial
  Primer
gaaattaatacgactcactatagggagaccacaacggtttccctctagaaataattttgtttaactttaagaagg

SEQ ID NO: 7 - DNA, 47 bp, Artificial
  Primer
gaaattaatacgactcactatagggtttaactttaagaaggagatat

SEQ ID NO: 8 - DNA, 41 bp, Artificial
  Primer
caaaaaacccctcaagacccgtttagaggccccaagggggg

SEQ ID NO: 9 - DNA, 72 bp, Artificial
  Primer
caaaaaacccctcaagacccgtttagaggccccaaggggattatttttcgaactgcgggtggctccaagcgc

SEQ ID NO: 10 - DNA, 95 bp, Artificial
  Primer
caaaaaacccctcaagacccgtttagaggccccaagggggggagtagaatgttaaggattagattatttttcgaactgcgggtggctccaagcgc

SEQ ID NO: 11 - DNA, 17 bp, Artificial
  Primer
taatacgactcactata

SEQ ID NO: 12 - DNA, 19 bp, Artificial
  Primer
tcacgttgtaaaacgacgg

SEQ ID NO: 13 - DNA, 26 bp, Artificial
  Primer
ccggaattctaatacgactcactata

SEQ ID NO: 14 - DNA, 33 bp, Artificial
  Primer
tcgcgacccgggcaaaaaacccctcaagacccg

(19) United States
(12) Patent Application Publication
(10) Pub. No.: US 2017/0029828 A1
(43) Pub. Date: Feb. 2, 2017

(54) NUCLEIC ACID WHICH IS STABILIZED AGAINST DECOMPOSITION

(71) Applicant: SIEMENS BUILDING TECHNOLOGIES AG, Zurich (CH)

(72) Inventors: Helmut Merk, Berlin (DE); Wolfgang Stiege, Berlin (DE)

(21) Appl. No.: 15/292,633

(22) Filed: Oct. 13, 2016

Related U.S. Application Data
(63) Continuation of application No. 14/804,818, filed on Jul. 21, 2015, now abandoned.
Application No. 14/804,818 is a continuation of application No. 14/551,696, filed on Nov. 24, 2014, now abandoned.
Application No. 14/551,696 is a continuation of application No. 13/922,393, filed on Jun. 20, 2013, now abandoned.
Application No. 13/922,393 is a continuation of application No. 13/223,427, filed on Sep. 1, 2011, now abandoned.
Application No. 13/223,427 is a continuation of application No. 10/471,936, filed on Apr. 29, 2004, now abandoned.

(30) Foreign Application Priority Data
Mar. 16, 2001 (DE) 10113265.4
Mar. 16, 2001 (DE) 10145014.1
Oct. 5, 2001 (DE) 10151071.3

(51) Int. Cl. C12N 15/68; C12P 19/34; C07K 14/47; C12P 21/00

(52) U.S. Cl. CPC C12N 15/68; C12P 21/00; C12P 19/34; C07K 14/4703

(57) ABSTRACT

The invention relates to a nucleic acid which is stabilised against decomposition by exonucleases. Said nucleic acid contains the following constituents: a) a code sequence coding for a defined protein, b) optionally, a promoter sequence controlling the expression of the code sequence, and c) at least one molecule A added to an end of the linear sequence containing the constituents a and b, said molecule being linked to a non-immobilised, volumic molecule B.

### CROSS REFERENCE TO RELATED APPLICATIONS

This application is a continuation of Ser. No. 14/804,818, filed Jul. 21, 2015, which is a continuation of U.S. application Ser. No. 14/551,696, filed Nov. 24, 2014, which is a continuation of U.S. application Ser. No. 13/922,393, filed Jun. 20, 2013, which is a continuation of U.S. application Ser. No. 13/223,427, filed Sep. 1, 2011, which is a continuation of U.S. application Ser. No. 10/471,936, filed Apr. 29, 2004, which is a National Stage Entry of PCT/DE02/01048, filed Mar. 18, 2002, which claims priority of DE 101 13 265.4, filed Mar. 16, 2001, which applications are incorporated herein by reference.

### SCOPE OF THE INVENTION

The invention relates to a nucleic-acid which is stabilised against decomposition, a method for producing such nucleic-acids as well as their application. Nucleic-acids may be DNA or RNA, but also PNA, single-stranded, or double-stranded.

### BACKGROUND OF THE INVENTION

Bioengineering and medical applications require proteins of high quality and quantity—measured on a gram and milligram scale. As far as larger proteins are concerned, classic synthesis is hardly possible and, in any event, uneconomical.

One possible means of producing proteins in large volumes is genetic engineering. For this purpose, cloned DNA, coded for the required protein, is inserted into cells, particularly procaryontic cells, as foreign DNA in the form of vectors or plasmids. These cells are then cultivated, whereby the proteins coded by the foreign DNA are expressed and extracted. Although this method allows the gain of considerable amounts of protein, the measures known, in particular cloning, are still costly. Furthermore, the cells are usually only transiently transfected and only exceptionally stably immortalised. A continuous production of protein thus requires a steady supply of fresh cells, which in turn have to be produced using the above described costly measures.

A further approach is the so-called cell-free in-vitro protein biosynthesis. This method applies biologically active cell extracts that are to a large extent free of the naturally occurring cellular nucleic-acid, and which are spiked with amino acids, energy-supplying substances and at least one nucleic-acid. The added nucleic-acid does the coding for the protein that is to be produced. When DNA is applied as the nucleic-acid, a DNA-dependent RNA polymerase must be present. Of course, RNA, mRNA can also be applied directly. By means of this approach it is not only possible to produce quickly and with comparably moderate costs such proteins that could also be produced genetically, but rather, it is possible to produce such proteins that are, for example, cytotoxic and thus not expressible to any considerable degree with the usual genetic engineering systems. However, in this case the manufacturer must produce the added nucleic-acid himself, a process which is then again costly by genetic engineering methods. To improve the efficiency of a protein synthesis it is often additionally desirable to introduce regulatory sequences and other sequences such as spacers, which are not naturally linked with a protein sequence.

An alternative to the genetically engineered production of complete nucleic-acids applicable in cell-free protein synthesis is the so-called expressions PCR. Here the efficient introduction of regulatory sequences (as well as other sequences promoting translational efficiency) into a nucleic-acid to be produced plays a special role within the framework of amplification. To introduce such further sequences into a target nucleic-acid, it is necessary to have very long PCR primers. However, on the one hand it is costly to produce long primers while, on the other hand, their application increases the probability of generating inhomogeneous PCR products.

Independent of the method used to produce nucleic-acids for cell-free protein biosynthesis, the following basic difficulty arises. Within the framework of this method of synthesis, so-called cytolysates i.e. extracts from cells, which contain the essential components and cell elements for protein synthesis, are used. However, the application of such cytolysates requires that the (exo-) nucleases naturally existing in the original cells are, as it were, transported into the lysate. These nucleases cause decomposition of the nucleic-acids produced for the protein synthesis, and thus reduce their half-life and consequently the protein exploitation. For obvious reasons this is a disturbing factor. Naturally, the same difficulty arises in the case of cellular systems.

### BRIEF DESCRIPTION OF THE DRAWINGS

FIG. 1 shows the primers that were used in the present invention as follows:
Primer A1 (SEQ.ID.NO: 1)
Primer A2 (SEQ.ID.NO: 2)
Primer B1 ((SEQ.ID.NO: 3)
Primer B2 (SEQ.ID.NO: 4)
Primer B3 (SEQ.ID.NO: 5)
Primer C1 (SEQ.ID.NO: 6)
Primer C2 (SEQ.ID.NO: 7)
Primer D1 (SEQ.ID.NO: 8)
Primer D2 (SEQ.ID.NO: 9)
Primer D3 (SEQ.ID.NO: 10)
Primer P1 (SEQ.ID.NO: 11)
Primer P2 (SEQ.ID.NO: 12)
BIOF (SEQ.ID.NO: 13)
BIOR (SEQ.ID.NO: 14)

FIG. 2 is a schematic representation of a single-stage PCR according to the invention, with four primers.

FIG. 3 shows that all sequence ranges except 0 (lower curve) have the effect of increasing protein synthesis.

FIG. 4 shows that synthesis can be improved with the Phage T7 Gen 10 transcription terminator by a factor of at least 2.8.

FIG. 5 shows that the spacer sequence results in an approximately 2-fold increase in expression.

FIG. 6 shows the half-life of the PCR product is approx. 100 min., corresponding to the course of the H-FABP synthesis running up into a plateau.

FIG. 7 shows the structure of Biotin, Biotin linked to Streptavidin, and Biotin and Streptavidin decomposition as a function of time.

FIG. 8 shows the influence of Streptavidin on protein synthesis.

### STATE OF THE ART

The use of cytolysates containing small amounts of natural (exo-) nucleases is in practice known within the framework of protein synthesis. An example of this is the Escherichia coli S-30 lysate. Although, compared with other lysates, its application provides an improved half-life of the intact nucleic-acid within the synthesis system, and thus an increase in protein exploitation using the same amount of nucleic-acid, there is still a disturbing amount of decomposition caused by nuclease.

In practice it is also known that, for purification purposes, the end of a nucleic-acid can be provided with an affinity molecule, for example Biotin. Biotin is then in a position to link to an immobilised Streptavidin, which causes the nucleic-acid to become immobilised, separated from the solution and its other components, and then—purified—to become detached again by the solid phase.

### Technical Problem of the Invention

The invention is based on the problem of determining nucleic-acids which, when used in a cell-free protein-synthesis system, provide an improved half-life and consequently an improvement in protein exploitation.

### Essential Features of the Invention

To solve this problem, the invention teaches a nucleic-acid that has been stabilised against decomposition with exonucleases, and having the following components: a) a code sequence coding for a defined protein, b) a promoter sequence controlling the expression of the code sequence, c) at least one molecule A added to an end of the linear sequence containing the constituents a and b, said molecule being linked to a non-immobilised volumic molecule B. Volumic molecules B may include ones that have a molecular weight of more than 500, preferably more than 1000, more preferably more than 10000. Appropriately such volumic molecules B will be proteins. Molecules A will be comparably small, having at least one binding site for a molecule B paired to the molecule A. Molecule B may have one or more binding sites for a molecule A paired to the molecule B. The link between a molecule A and a molecule B may be both non-covalent as well as covalent. The linking of molecule A to a terminal nucleotide of the linear sequence is appropriately covalent. When one molecule A each is attached both to the 3′-end as well as the 5′-end of the linear sequence, the nucleic-acid according to the invention is stabilised with respect to both 3′-as well as 5′-exonucleases. It is possible to attach molecules A to both ends by hybridizing a primer with molecule A to the 5′-end of both the sense strand as well as the antisense strand of a double-stranded nucleic-acid.

The invention is based on the knowledge that modified primers, i.e. such primers that are carrying a molecule A, can be used to produce nucleic-acids in large volumes and by simple means using PCR, whereby these nucleic-acids will be carrying a molecule A on at least one end. A volumic protective group for preventing an attack by exonucleases can then be simply attached by means of molecule B. The required amount of molecules B can be applied without difficulty, because commercially available and inexpensive proteins can be used for this purpose.

Where a molecule B has several binding sites n for a molecule A paired to the molecule B, it may be appropriate to saturate a number of the binding sites, particularly n−1 binding sites, in such a way that only few molecules A, particularly only one molecule A will link to the molecule B. To saturate the binding sites on molecule B, it is possible to use molecules A not linked to primers as well as other molecules which link to molecule B's binding sites for molecules A. It is clear that the required high volume of molecules B will be used in the solution, so that in spite of the saturation that is accomplished, each molecule A can bind one molecule B. By saturating the binding sites on molecule B, that are necessary for the links of molecule B, it is possible to prevent a molecule B from linking several molecules A, which would lead to an aggregation of molecule-A-linked-primers on molecule B, which in turn could disturb the amplification of the nucleic-acid base sequences. Such a situation could also cause a clustering of several amplified nucleic-acid base sequences via the molecules A attached to a molecule B with the primers, which would render more difficult the transcription and/or translation of the nucleic-acid base sequences and thus reduce the translated quantity of protein coded by the nucleic-acid sequence.

To reduce, and in particular to prevent the clustering of several amplified nucleic-acid base sequences via the molecules A attached to the molecule B with the primers, it may be of advantage to perform the transcription and/or translation directly after the amplification, with respect to time.

The invention achieves that the exonucleases can no longer attack and decompose the nucleic-acids produced and applied in protein synthesis, or that they can do so only on a much reduced scale. The result of this is that the half-life of the elaborately and thus costly produced nucleic-acids is considerably increased in an expression system, so that a corresponding increase in protein exploitation is accomplished with the same or even less quantitative input of nucleic-acids.

The invention further teaches a method for producing a nucleic-acid according to the invention, by using the following production steps: 1) a linear sequence with the constituents a) and b) is produced; 2) the linear sequence from step 1) is amplified by PCR, whereby at least one primer or a primer pair is used which carries the molecule A; 3) the product from step 2) is incubated with a solution containing molecule B. A particularly advantageous embodiment of such a method is a method of preparation of long nucleic-acids by means of PCR, using the following steps of hybridisation: a) a nucleic-acid base sequence is hybridised to the 3′-end and the 5′-end using an adapter primer in each case; b) the product from step a) is hybridised to the 3′-end and the 5′-end using an extension primer that contains an extension sequence, whereby a nucleic-acid sequence is formed from this nucleic-acid base sequence extended and amplified by extension sequences attached to the 3′-end and the 5′-end of the nucleic-acid base sequence, and whereby preferably the primers applied in a last amplification stage carry a molecule A.

A nucleic-acid base sequence is a sequence that codes for a protein. This may in particular be a gene, but may also consist of sequences made up of genomes without introns. The extension sequences may in particular be sequences that encompass a regulatory sequence and or sequences that contain a ribosomal linking sequence. Adapter primers are comparably short. One part of an adapter primer is specific for the nucleic-acid base sequence, while another part is constant and hybridises one extension sequence respectively.

From this it follows that it is not necessary to apply “matching” long extension sequences for each different type of nucleic-acid base sequence. Rather, it is adequate to co-ordinate the comparatively short adapter primer to a defined nucleic base sequence, while the extension sequences may as it were be universal, i.e. for different nucleic-acid base sequences it is possible to always use the same or a few selected extension sequences, as the case may be. Thus the relatively expensively produced extension sequences may be provided for a wide range of applications, while for a specific nucleic-acid base sequence it is merely necessary to produce the adapter sequences. The latter require little expenditure because the adapter sequences may be quite short.

For example, this makes it possible that both a regulatory sequence as well as a ribosomal linking sequence can be linked to a nucleic-acid base sequence, each via an extension primer, and this may even be done within a PCR step. It is thus possible to obtain a nucleic-acid that results in a particularly high level of transcription efficiency and/or translation efficiency within one procaryontic system of cell-free protein synthesis.

A particular advantage of this embodiment of the invention is that it is a generally applicable method for any coding sequences.

Finally, the invention teaches the use of a nucleic-acid according to the invention within a method for producing a protein coded by the code sequence within a cell-free protein biosynthesis system or within a cellular protein biosynthesis system. With respect to the method steps for cell-free protein synthesis, reference is made to the embodiment examples, from which a specialist can easily generalize the fundamental characteristics.

### Embodiments of the Invention

With respect to the nucleic-acid according to the invention, it is preferred that both ends of the linear sequence are linked with one molecule A each, as this will then ensure a complete stabilisation of both ends of the sequence with respect to exonucleases.

In order to prevent the expression from being obstructed by volumic molecules B, it may be recommendable to establish a spacer sequence between constituents a and/or b and molecule A.

Specifically, each molecule A can be respectively linked to one molecule B, or both molecules A can be linked to a single molecule B having at least two binding sites for a molecule A. In the former case, a linear product is produced. In the latter case a circularised product, which can be improved with respect to stability, is produced.

Molecule A may be Biotin or Digoxigenin, and molecule B can be Avidin, Streptavidin or Anti-Digoxigenin antibody. These are commercial products available in large quantities and at low cost.

In the case where molecule B is Avidin or Streptavidin, it may be appropriate to saturate a part of the n binding sites, particularly n−1 binding sites, in such a way than only a few Biotin molecules, particularly only one Biotin molecule can link to an Avidin molecule or a Streptavidin molecule. To saturate the binding sites of Avidin or Streptavidin, it is possible to use Biotin not linked to primers as well as other molecules which link to the binding sites of Avidin and Streptavidin. It is clear that the required high volume of Avidin and/or Streptavidin will be used in the solution, so that in spite of the saturation that is accomplished, each Biotin molecule can bind one Avidin or Streptavidin molecule. By saturating the binding sites of Avidin or Streptavidin for the Biotin links, it is possible to prevent an Avidin molecule or Streptavidin molecule from linking several Biotin molecules, which would lead to an aggregation of Biotin-linked-primers on an Avidin molecule or Streptavidin molecule, which in turn could disturb the amplification, transcription or translation of the nucleic-acid base sequences.

Within the framework of producing a nucleic-acid according to the invention, a further development is of independent significance, whereby the product from step b) can within a step c) be hybridised to the 3′-end and the 5′-end with one amplification primer, respectively, whereby an amplified nucleic-acid end sequence is formed. It is clear that the primers of step c) are then provided with the molecule A. The amplification primers too are on the one hand comparably short and universally applicable and thus readily available. By means of the amplification primers it is additionally possible to attach further (shorter) sequences to the ends, which would then further increase the translation efficiency. By means of the short amplification primers it is possible to introduce other variations and modifications to the ends of the nucleic-acid without much expense. This is of particular advantage because it is not necessary to produce or use different extension primers for variations and modifications, which would otherwise be necessary to an unfavourable extent.

In particular, according to the invention a Biotin residue may be connected to the 5′-end of the amplification primer. Following incubation of the nucleic-acid end sequence with Biotin-linking Streptavidin, this provides a nucleic-acid end sequence stabilised against exonuclease-decomposition, which leads to a multiple increase in the half-life of an in-vitro protein synthesis system as compared with a non-stabilised nucleic-acid end sequence, typically a 5-fold increase, for example from 15 min. to approx. 2 hours. The stabilities accomplished for linear constructs are comparable to those of classic circular plasmids, and insofar they can practically replace these equivalently.

It may be noted that a molecule A may also have a double or multiple function, for example it may simultaneously function as an anchor group.

The adapter primers typically contain <70, in particular 20-60 nucleotides. The extension primers typically contain ≧70, even 90 and more nucleotides. The amplification primers on the other hand typically contain <70, usually <30 nucleotides, typically >9 nucleotides. It is only necessary for the adapter primers to be specifically adapted to a defined nucleic-acid base sequence, which, in the light of the relatively short sequences required, involves little cost.

Advantageously, steps a), b) and optionally step c) are performed in a PCR solution containing the nucleic-acid base sequence, the adapter primers, the extension primers and optionally the amplification primers. It is then a single-stage PCR with a total of 6 primers, two adapter sequences, two extension sequences and two amplification sequences. It is then adequate to apply low concentrations of the adapter primers and extension primers, so that only low quantities of the intermediate product are produced. Furthermore, the intermediate product does not need to be homogeneous, so that elaborate optimisations are not required. Due to the shortness of the amplification primers, even with amplification to high quantities of nucleic-acid end sequences no optimisations are required.

Alternatively to the above embodiments, a variation operating with two PCR stages is independently significant. In such an embodiment, steps a) and b) are performed a defined first number of cycles in a process stage A) in a pre-PCR solution containing the nucleic-acid base sequence, the adapter primers and the extension primers, while step c) is performed a defined second number of cycles in a process stage B) in a main PCR solution containing the PCR product from stage A) and the amplification primers. It is thereby possible to perform stage A) with a reaction volume that is ½ to 1/10 of the volume of stage B). In stage A), the lower volume will then lead to a higher concentration of the intermediate product or rather it is possible to apply considerably less nucleic-acid base sequence. By means of dilution with the PCR solution volume in the transition from stage A) to stage B), the adapter primers and the extension primers in turn are strongly diluted, with the result of an increased probability that the variations and/or modifications will be inserted into the nucleic-acid end sequences via the amplification primers.

Specifically, in the first of the above alternatives it is possible to proceed such that the PCR is performed with a reaction volume of 10 to 100 μl, preferably 20 to 40 μl, with 0.01 to 100 pg, preferably 1 to 50 pg nucleic-acid base sequence, 0.05 to 10 μM, preferably 0.1 to 5 μM adapter primer and 0.005 to 0.5 μM, preferably 0.001 to 0.1 μM extension primer, whereby, following a defined initial number of cycles 0.01 to 10 μM, preferably 0.1 to 10 μM of amplification primer are added, and whereby the amplified nucleic-acid end sequence is then subsequently produced via a defined number of successive cycles. The following reaction conditions are recommended for the above second alternative: stage A): reaction volume <10μl; 0.001 to 50 pg, preferably 0.01 to 5 pg nucleic-acid base sequence; 0.05 to 10 μM, preferably 0.1 to 5 μM adapter primer, and 0.05 to 10 μM, preferably 0.1 to 5 μM extension primer; initial number of cycles 10 to 30, preferably 15 to 25; stage B): reaction volume 10 to 100 μl, preferably 15 to 50 μl, maintained by supplementing the solution from stage A) with PCR solution; 0.01 to 10 μM, preferably 0.1 to 5 μM amplification primer; second number of cycles 15 to 50, preferably 20 to 40.

Nucleic acids according to the invention are, for example, applicable for cell-free in-vitro protein biosynthesis, particularly in procaryontic systems, preferably in a translation system of Escherichia coli D10.

Utilisation according to the invention is advantageously applicable for the selective amplification of a defined nucleic-acid base sequence from a nucleic-acid library. This facilitates a characterisation of gene sequences, whereby the gene sequence is applied as a nucleic-acid base sequence and whereby the protein obtained is analysed with respect to its structure and/or function. The background of this aspect is that, although the sequences of many genes are known, the structure and function of the thereby coded protein is not known. Thus the elements of a gene library, for which only the sequence may be known, can be examined with respect to its function within an organism. The examination of the structure and function of the protein obtained is then performed using the usual methods of work applied in biochemistry.

By means of the method according to the invention, it is possible to gain nucleic acids that contain a coding nucleic-acid base sequence for a protein and a ribosomal linking sequence as well as one or more sequences from a group consisting of “promoter sequence, transcription terminator sequence, expression enhancer sequence, stabilising sequence and affinity tag sequence”. An affinity tag sequence codes for a structure that has a high affinity for (usually immobilised) binding sites in separating systems for purification. This facilitates an easy and highly affine separation of proteins that do not contain the affinity tag. An example of this is Strep-tag II, a peptide structure of 8 amino-acid residues with affinity to StrepTactin. A stabilising sequence codes for a structure that is either itself stable against decomposition, or becomes stable against decomposition after linking to a linking molecule that is specific for the structure, particularly by means of nucleases. Such a stabilising sequence can be attached to an end that is not provided with a molecule A. An expression enhancer sequence increases translation efficiency as compared with a nucleic acid without an expression enhancer sequence. These may be, for example, (non-translated) spacers. A transcription terminator sequence terminates the RNA synthesis. An example of this is the T7 Phage gene 10 transcription terminator. Transcription terminator sequences can also provide stabilisation against decomposition through 3′-exonucleases. Advantageous relative arrangements of the above sequence elements to each other can be generalised from the following embodiment examples.

The following examples are merely preferred examples that serve to further explain the invention.

### Methods:

PCR: The PCR was performed in a reaction volume quantified in the examples with 10 mM Tris-HCl (pH 8.85 at 20° C.), 25 mM KCl, 5 mM (NH₄)₂SO₄, 2 mM MgSO₄, 0.25 mM each dNTP, 3 U Pwo DNA polymerase (Roche) as well as the amounts of nucleic-acid base sequences specified in the examples. The cycles were performed for 0.5 min. at 94° C., 1 min. at 55° C. and 1 min. at 72° C.

In-Vitro Expression:

In-vitro experiments were performed in compliance with the literature information given in Zubay, G.; Annu. Rev. Genet. 7:267-287 (1973) with the following modifications. The Escherichia coli S-30 lysate was supplemented with 750 U/ml T7 Phagen RNA polymerase (Stratagene) and 300 μM [¹⁴C]Leu (15 dpm/pmol, Amersham. PCR products and control plasmids were used in concentrations of 1 nM to 15 nM. The reactions were performed at 37° C., whereby the course of the reactions was followed by means of 5 μl aliquots being taken from the reaction mixture at successive points in time, and the insertion of [¹⁴C]Leu was estimated by TCA precipitation. Further 10 μl aliquots were taken for the purpose of analysis of the synthetic protein by means of SDS-PAGE, followed by an autoradiography in a phospho-imager system (Molecular Dynamics).

Plasmid Construction:

A high-copy derivate of the plasmid pET BH-FABP (Specht, B. et al.; J. Biotechnol. 33:259-269 (1994)), which codes for bovine heart fatty acid binding protein, was constructed, called pHMFA. A fragment of pET-FABP was produced by digestion with endonucleases SphI and EcoRI and inserted into vector pUC18. With respect to the sequences that are relevant for the synthesis of H-FABP, the plasmid pHMFA is identical to the original plasmid. It is noted that the linearised plasmid does not behave any better than the circular plasmid.

Construction of Nucleic Acids with Various Sequence Ranges Upstream of the Promoter:

The pHMFA plasmid served as a template for constructing nucleic acids with different sequence ranges upstream of the promoter. The constructs (see examples) FA1, FA2 and FA3 with 0, 5 and 249 base pairs upstream of the promoter were generated with primers P1, C1 and P2 as well as with the downstream primer P3. Construct FA3 with a sequence range of 15 base pairs upstream of the promoter was obtained by digestion of FA4 with endonuclease Bgl II. The control plasmid pHMFA (EcoRV) with a sequence range of 3040 base pairs was obtained by digestion of the plasmid with EcoRV. All products were purified by Agarose Gel Electrophoresis, followed by gel extraction using the “High Pure PCR Product Purification Kit”.

Affinity Purification:

Purification of the fatty acid binding protein containing Strep-tag II (Voss, S. et al.; Protein Eng. 10:975-982 (1997)) was performed by affinity chromatography as per manufacturer instructions (IBA Gottingen, Germany), a deviation being that the volume of the affinity column was reduced (200 μl). The reaction mixture of the connected transcription/translation was briefly centrifuged and then applied to the column. Isolated fractions were analysed by TCA precipitation and autoradiography by means of SDS-PAGE (see above).

H-FABP Activity Assay:

The complete reaction mixture with in-vitro synthesised H-FABP was investigated with respect to the activity of the linking of oleic acid. Different volumes (0-30 μl) were filled up to 30 μl with reaction mixture without H-FABP and diluted with translation buffer (50 mM HEPES pH 7.6, 70 mM KOAc, 30 mM NH₄Cl, 10 mM MgCl₂, 0.1 mM EDTA, 0.002% NaN₃) to a final volume of 120 μl. After the addition of 2 μl 5 mM [9,10(n)-³H] oleic acid (Amersham) with a specific activity of 1000 dpm/pmol, the specimens were incubated for one hour at 37° C. 50 μl of the specimens were used to remove uncombined oleic acid by means of gel filtration (Micro Bio Spin Chromatography; Bio-Rad). The ³H radioactivity of the eluted fractions was measured by means of a scintillation counter.

Analysis of the Stability of Nucleic Acids:

Radioactively marked nucleic acids were synthesized in accordance with the above conditions, however in the presence of 0.167 μCi/μl [α-³⁵S] dCTP. The marked nucleic acids were applied in a connected transcription/translation, reaction volume 400 μl. 30 μl aliquots were taken at successive points in time. After adding 15 μg ribonuclease A (DNAse-free, Roche) these were incubated for 15 min. at 37° C. After addition of 0.5 SDS, 20 mM EDTA and 500 μg/ml proteinase K (Gibco BRL) to provide a total reaction volume of 60 μl, further incubation for 30 min. at 37° C. was performed. Residual PCR products were further purified by ethanol precipitation and were then subjected to a denaturalising electrophoresis (5.3% polyacrylamide, 7 M urea, 0.1% SDS, TBE). The dried gel was allowed to run through a phospho imager system (Molecular Dynamics) for quantification.

Sequences:

FIG. 1 shows the primer sequences that were used.

### Example 1

### PCR with 4 Primers

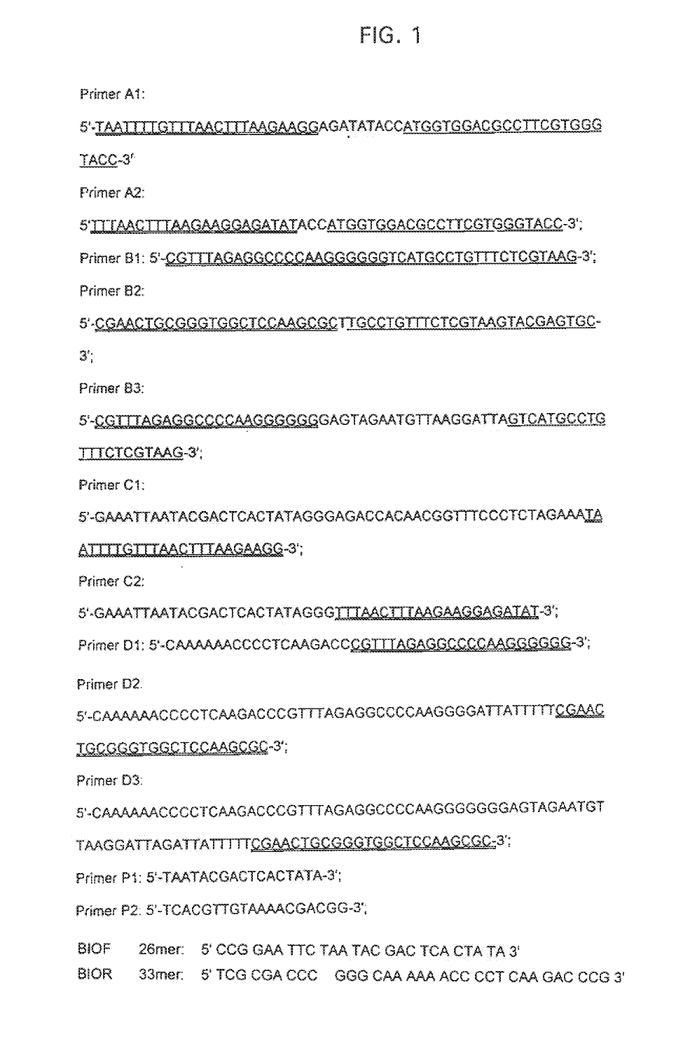

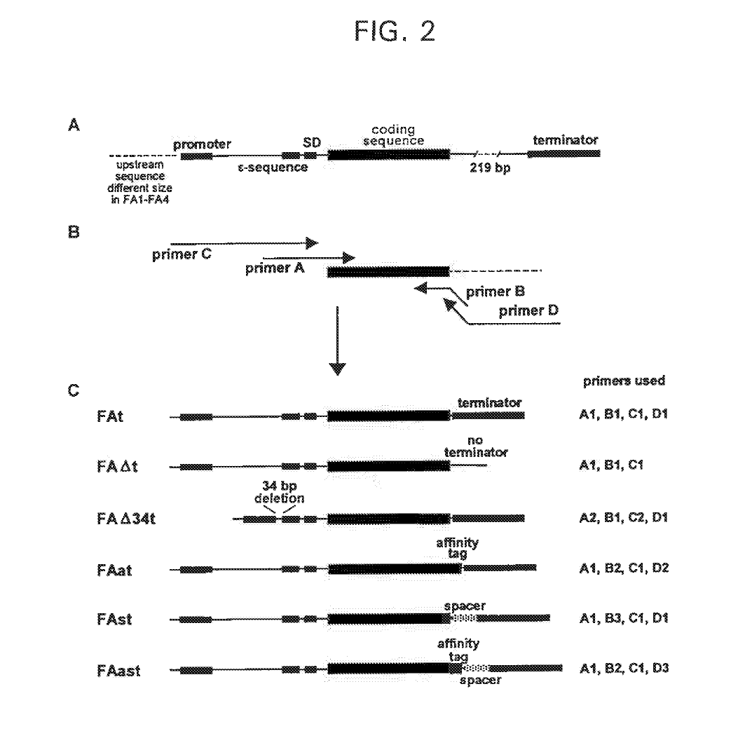

FIG. 2 is a schematic representation of a single-stage PCR according to the invention, with four primers. In the middle can be seen the nucleic-acid base sequence coding for a protein, which encompasses the complete coding sequence for H-FABP (homogeneous and functionally active fatty acid binding protein from bovine heart), obtained as a 548 bp restriction fragment of pHMFA by digestion with the endonucleases Ncol and BamHI (as well as a 150 bp sequence at the 3′-end, which is neither translated nor is it complementary to an adapter primer or an extension primer). This is where the two adapter primers A and B are hybridised, which with the ends of the nucleic-acid base sequence encompass homologous ends. Adapter primer A furthermore contains a ribosomal linking sequence. The extension primers C and D are hybridised to the outer ends of adapter primers A and B. Extension primer C encompasses the T7 Gen 10 leader sequence including the T7 transcription promoter as well as an optional sequence upstream consisting, for example, of 5 nucleotides. The extension primer D encompasses the T7 Gen 10 terminator sequence.

### Example 2

### Efficiency of H-FABP Synthesis in Dependence of the Sequence Range Upstream of the Promoter

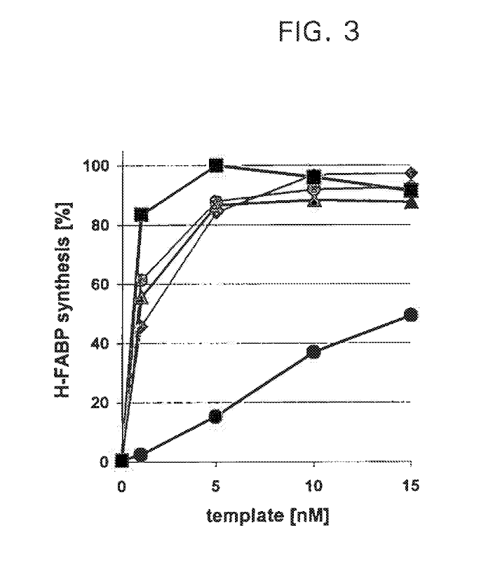

Four PCR products (FA1 through FA4) with different sequence ranges upstream of the promoter (0, 5, 15, 250 base pairs) and the linearised control plasmid pHMFA(EcoRV) with 3040 bp upstream of the promoter were investigated for in-vitro transcription/translation at different concentrations (1, 5, 10 and 15 mM). FIG. 3 shows that all sequence ranges except 0 (lower curve) have the effect of increasing protein synthesis. Even 5 base pairs are adequate.

### Example 3

### Improvement of H-FABP Synthesis by Means of Phage T7 Gen 10 Transcription Terminator/5′ Leader Sequence Phage T7 Gen 10

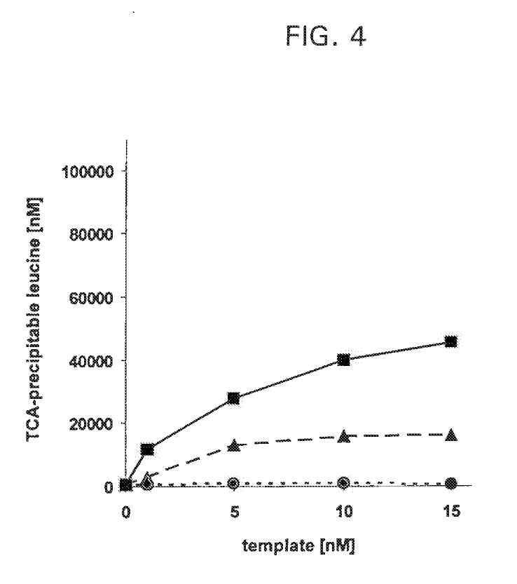

FIG. 4 shows that synthesis can be improved with the Phage T7 Gen 10 transcription terminator by a factor of at least 2.8. The triangles represent FAΔt, while the squares represent FAt (see also FIG. 2).

Furthermore, FIG. 4 shows that a deletion of 34 bp between the transcription-start and the epsilon sequence (Olins, P. O. et al.; Escherichia coli. J. Biol. Chem. 264:16973-16976 (1989) leads to a suppression of product formation. The circles represent this variation FAΔ34 (see also FIG. 2).

### Example 4

### Influence of the Position of the Transcription Terminator Sequence

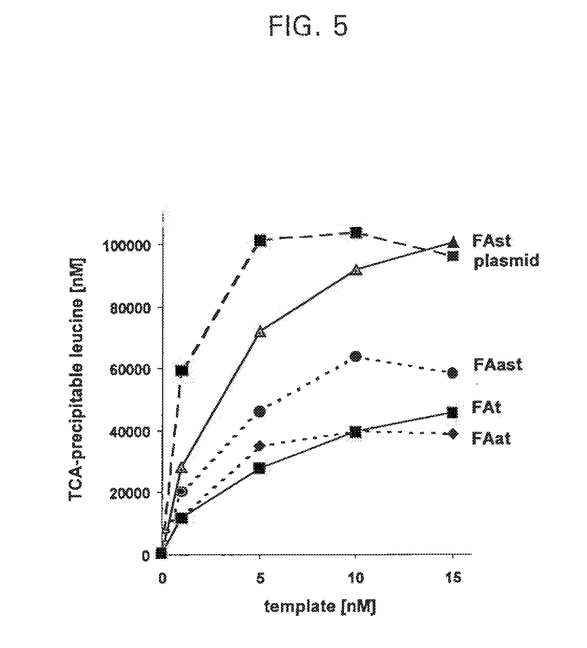

Products FAst and FAast (see FIG. 2) were produced for the purpose of investigating the influence of the position of the terminator sequence. Both are identical to FAt and FAat, except that a 22 bp spacer sequence was introduced between the stopcodon and the terminator by means of different primers. FIG. 5 shows that the spacer sequence results in an approximately 2-fold increase in expression.

By comparing FAt and FAat in FIG. 5 it can, however, also be seen that an affinity tag hardly has an influence on the expression.

### Example 5

### PCR Out of a Complex DNA Mixture

The effectiveness and specificity of the method according to the invention was examined in the presence of a large amount of competitive DNA. A PCR was performed for FAst according to the above description, but with the following exceptions: the nucleic-acid base sequence was used in concentrations of 0.16 to 20 pg/50 μl reactor volume, and the reactions were supplemented with 0.83 μg chromosomal DNA of Escherichia coli, ultrasonically treated for 5 min. It was found that neither the quality nor the quantity of the PCR product was influenced by the presence of the 5 million-fold excess of competitive DNA.

### Example 6

### Affinity Purification with Strep-Tag II

A reaction mixture with 10 μg of the radioactively marked FAast was subjected to affinity purification. Approximately 81% of the applied material was maintained by the column and 67% were gained as a pure product in the elution fraction (calculated from TCA precipitation of the fractions of the affinity column.

### Example 7

### Activity of the PCR Product

Samples of H-FABP, synthesised either by means of the plasmid or as the PCR product FAast, were investigated together with respect to linking activity for oleic acid. Following transcription/translation, different volumes with 0 to 330 pmol of non-marked H-FABP were examined in a linking assay according to the above description on methods. The activities were found to be identical, independent of the method of production.

### Example 8

### Stability of the PCR Product

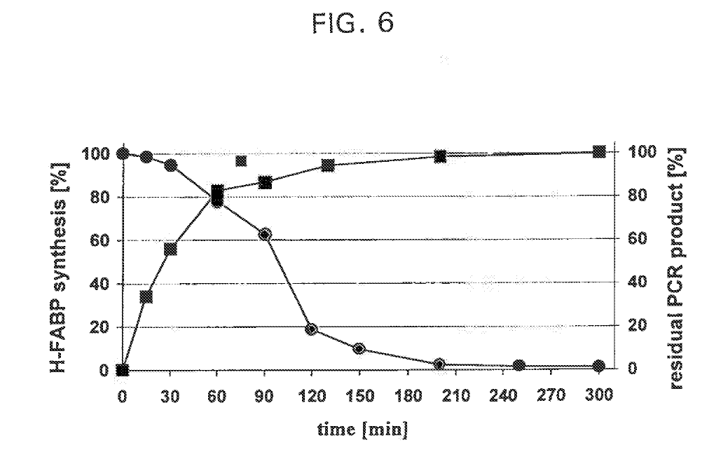

The reduction of the PCR product FAast was measured to determine whether the stability of the PCR possibly restricts the effectiveness of the expression. The radioactively marked product was used for this. Aliquots of the reaction mixture were taken at certain time intervals and then examined with denaturalising polyacrylamide-gel-electrophoresis. The quantity on the remaining PCR product was quantified by scanning the radioactivity of the gel and compared with the time response of the protein synthesis, measured by scanning the radioactivity of H-FABP in the gel after separating the reaction mixtures by means of SDS-PAGE. The results are shown in FIG. 6. It can be seen that the half-life of the PCR product is approx. 100 min., which corresponds to the course of the H-FABP synthesis running up into a plateau.

### Example 9

### Optimised Conditions for a PCR with Four Primers

Table I shows the optimised conditions for a PCR with four primers in a reaction volume of 25 μl.

TABLE I
a) Reaction components

| | Concentration |
|---|---|
| Reaction components | in reaction |
| PCR buffer for Pwo Polymerase (Roche) | according to |
| | manufacturer |
| Desoxynucleotide triphosphate dATP, dCTP, dGTP and | 0.25 mM |
| dTTP | |
| Adapter primer a (55 nucleotides) | 0.1 μM |
| Adapter primer b (51 nucleotides) | 0.1 μM |
| Extension primer c (75 nucleotides) | 0.4 μM |
| Extension primer d (95 nucleotides) | 0.4 μM |
| Template: coding sequence for fatty acid binding protein | 10 pg/25 μl |
| restriction fragment from pHM18FA (Ncol/BamHI): | |
| Pwo DNA polymerase (Roche) | 1.5 U/25 μl |

b) Temperature program
Temperature cycle

| | | |
|---|---|---|
| Segment 1 | 30 sec | 94° C. |
| Segment 2 | 60 sec | 55° C. |
| Segment 3 | 60 sec | 72° C. |

60 cycle repetitions

### Example 10

### PCR with 6 Primers

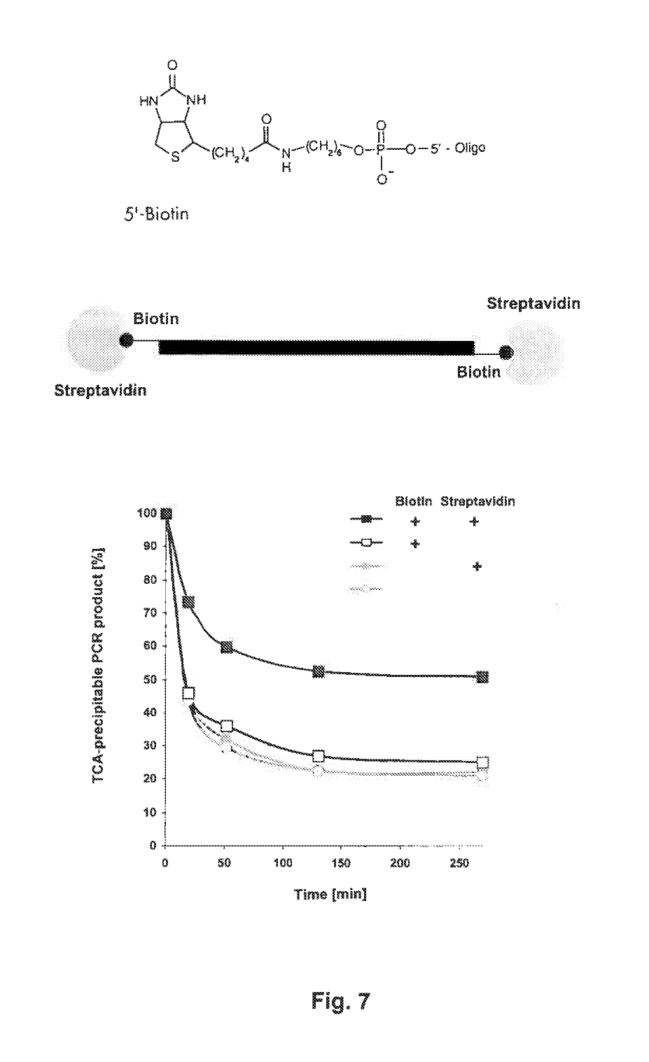

Varying extension primers were set up using the materials from example 9, however with two additional amplification primers e (26 nucleotides) and f (33 nucleotides) as well as an increased adapter primer concentration of 0.2 μM. Reference is made to FIG. 1 with respect to the amplification primers—BIOR and BIOF there. BIOF is a Biotin marked forward primer, and BIOR is a Biotin marked reverse primer. The structure is represented in FIG. 7.

A minimum requirement for expensive extension primer resulted when initially 25 cycles were run without amplification primer followed by a further 25 cycles run with amplification primer. By using the amplification primer it was possible to reduce the concentration of extension primer down to 0.025 μM, a factor of approx. 1/20, while still accomplishing improved homogeneity and exploitation of the PCR product.

These advantages are based on the fact that the use of the six primers strongly reduces the probability of intermediate products forming, because the primers, which are necessary for intermediate product formation, are used in low concentrations. Intermediate products can thus not be concentrated exponentially with the amplification primers.

### Example 11

### PCR with Six Primers and Two Stages

Principally, the materials specified above are used. Initially, a pre-PCR is performed in a reaction volume of 5 μl with 0.1 pg nucleic-acid base sequence, and using 0.3 μM adapter primer and 0.5 μM extension primer through 20 cycles. The reaction solution obtained by these means is diluted with PCR volume to 25 μl. Then the amplification primer is added to a final concentration of 0.5 μM. Finally another 30 cycles is performed for amplification.

### Example 12

### Stabilising a Nucleic Acid with Biotin

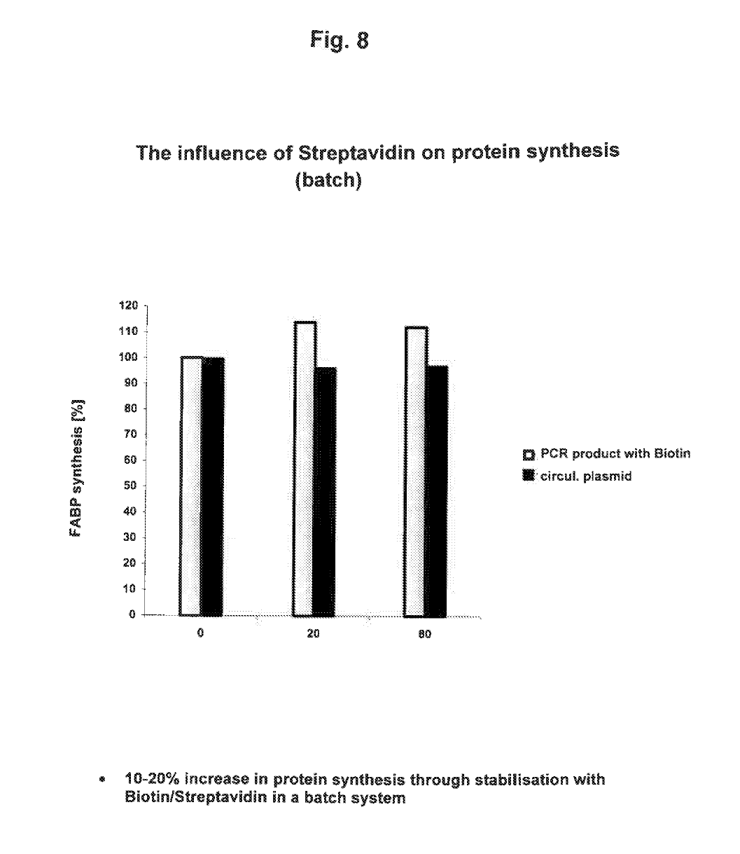

A nucleic acid was produced using primers BIOF and BIOR during the course of the PCR with 6 primers—as described above; both its decomposition as a function of time and the improvement in protein synthesis were studied. This is shown in FIGS. 7 and 8. It can be seen that, particularly after turnover with Streptavidin, a considerable improvement in stability is accomplished with Biotin. This also leads to a 20% increase in protein synthesis, even in a system with small amounts of exonucleases. The example thus proves that even in such systems, protein-synthesis performance is improved. In systems with lysates, which have higher levels of exonucleases, improvements in synthesis performance by a factor up to 5 and more can be expected.

Independent of the above described examples, it is to be noted that with the method according to the invention it is also possible to very easily have variations of sequences through mutations, for example by applying tag-polymerase and/or altered reaction conditions. If this is not required, work can be preferably carried out with Pwo or Pfu, which function more precisely and have proofreading activities.

1. A nucleic acid stabilised against decomposition by exonucleases and containing the following constituents:
a) a code sequence coding for a defined peptide or protein,
b) optionally, a promoter sequence controlling the expression of the code sequence, and
c) at least one molecule A added to an end of the linear sequence containing the constituents a and b, said molecule being linked to a non-immobilised, volumic molecule B.

2. The nucleic acid according to claim 1, whereby both ends of the linear sequence are linked to one molecule A each.

3. The nucleic acid according to claim 1, whereby a spacer sequence is arranged between the constituents a and/or b and the molecule A or the molecules A.

4. The nucleic acid according to claim 2, wherein either each molecule A is linked to a molecule B, or wherein both molecules A are linked to a single molecule B having at least two binding sites for a molecule A.

5. The nucleic acid according to claim 1, wherein the molecule A is Biotin or Digoxigenin and the molecule B is Avidin, Streptavidin or Anti-Digoxigenin Antibody.

6. A method for producing a nucleic acid according to claim 1 with the following process steps:
1) a linear sequence containing constituents a) and optionally b) is prepared,
2) the linear sequence from step 1) is amplified with PCR, whereby at least one primer or one primer pair is applied carrying molecule A,
3) the product from step 2) is incubated with a solution containing molecule B.

7. The application of nucleic acid according to claim 1 in a process for producing a protein coded by the code sequence in a cell-free protein biosynthesis system or in a cellular protein biosynthesis system.
